(19) United States
(12) Patent Application Publication
(10) Pub. No.: US 2009/0118457 A1
(43) Pub. Date: May 7, 2009

(54) Resorcinol-based mannich base

(75) Inventor: Ulrich Gerber, Uitikon-Waldegg (CH)

Correspondence Address: OLIFF & BERRIDGE, PLC, P.O. BOX 320850, ALEXANDRIA, VA, 22320-4850, US

(73) Assignee: SIKA TECHNOLOGY AG, Baar (CH)

(21) Appl. No.: 11/919,064

(22) PCT Filed: Apr. 28, 2006
(86) PCT No.: PCT/EP2006/061917

§ 371 Date: Mar. 24, 2008

(30) Foreign Application Priority Data
Apr. 29, 2005 (EP) 05103574.9

(51) Int. Cl. C07C 209/60; C08G 18/10

(52) U.S. Cl. 528/59; 564/280

(57) ABSTRACT

The invention relates to Mannich bases that can be produced from resorcinol, formaldehyde, and triethylenetetramine and/or tetraethylenepentamine. Also disclosed are a method for the production thereof and the use thereof as hardeners for amine-reactive compounds. The inventive Mannich bases are particularly suitable as hardener components in adhesives.

### TECHNICAL FIELD

The present invention relates to the field of the preparation and use of Mannich bases.

### PRIOR ART

The class of the Mannich bases has already been known for a long time and has already been employed in curing components of reactive systems. Phenols are employed for the preparation. Phenol (hydroxybenzene) as a starting material, however, has the great disadvantage that the Mannich bases prepared from it still contain fractions of unreacted phenol. Owing to the toxicity of phenol, phenol-based Mannich bases cannot be employed for numerous market segments. Great efforts have therefore been made to prepare phenol-free Mannich bases. Thus, for example, Mannich bases based on nonylphenol or p-tert-butylphenol or Cardanol have been developed and commercialized.

Mannich bases are employed primarily as accelerants for epoxy resins or as curing agents for epoxy resins and polyurethanes. WO 00/15687 describes, for example, a Mannich base accelerant prepared by transaminating a Mannich base with an amine.

The processes for preparing known Mannich bases are very inconvenient and difficult to carry out, particularly when the formation of high molecular mass condensation products is to be prevented as far as possible. Thus, for example, EP-A-1 475 411 discloses a two-stage preparation process for preparing Mannich bases based on m-cresol or 3,5-xylenol and polyamines, the process preferably using a tertiary amine. Another two-stage Mannich base preparation process is disclosed by EP-A-1 475 412, where said bases are obtained from phenols such as m-cresol, 3,5-xylenol or resorcinol with polyamines, preferably with the use of tertiary amines. Two-stage processes of these kinds, however, entail additional inconvenience and make Mannich base production more expensive.

### SUMMARY OF THE INVENTION

It is an object of the present invention, therefore, to provide new Mannich bases, and also the process for preparing them, which are free from phenol and can be prepared by a simplified process.

Surprisingly it has emerged that, through a specific selection of prior-art polyamine and phenolic compounds, Mannich bases according to claim 1 can be prepared that are able to achieve this object. These Mannich bases are preparable from inexpensive and readily obtainable raw materials via a simple preparation. They are notable for excellent cure behavior, in particular at low temperatures, with amine-reactive compounds.

Other aspects of the invention are described in the other main claims. Further advantageous embodiments of the invention are apparent from the dependent claims.

### EMBODIMENTS OF THE INVENTION

The present invention relates to Mannich bases which are preparable from resorcinol, formaldehyde and/or triethylenetetramine and tetraethylenepentamine.

Resorcinol (CAS No. [108-46-3]) is widely available commercially in different purities. One of the qualities which marks out resorcinol from the other dihydroxybenzene isomers, pyrocatechol and hydroquinone, is its lower toxicity (German water hazard class WGK 1 as against 2 or 3, or Swiss toxicity class 3 as against 2). It has emerged in particular that, as compared with these phenols and other phenols, such as phenol itself, for example, the various isomers of cresol or xylenol, resorcinol, surprisingly, is exceptionally well suited to the preparation of Mannich bases.

Formaldehyde in the forms known typically to the skilled worker can be employed directly or from formaldehyde donor compounds. Preference is given to formaldehyde in the form of para-formaldehyde or of formalin solution. Formalin solution is particularly preferred.

Also used for the preparation of the Mannich bases of the invention is triethylenetetramine and/or tetraethylenepentamine. Both triethylenetetramine (TETA) (CAS No. [112-24-3]) (3,6-diazaoctane-1,8-diamine) and tetraethylenepentamine (TEPA) (CAS No. [112-57-2]) (3,6,9-triazaundecane-1,1′-diamine) are widely available commercially and are very favorably priced. In particular they are available, and utilized, in technical grade. Technical grade of this kind is preferred. The skilled worker is aware that TETA and TEPA in this kind of technical grade is not a pure, chemically uniform substance. In particular, on account of the process for their preparation, they include further substances and isomers. The isomers and substances of this kind that have formed principally are,

### for TETA:

N,N′-bis(2-aminoethyl)piperazine (BisAEP or DiAEP) (CAS No. [6531-38-0])
piperazinoethylethylenediamine (PEEDA) (CAS No. [24028-46-4])
tris(2-aminoethyl)amine (NTEA or NTE) (CAS No. [4097-89-6])
   and, for TEPA:
4-aminoethyltriethylenetetramine (AETETA) (CAS No. [31295-46-2])
aminoethylpiperazinoethylethylenediamine (AEPEEDA) (CAS No. [31295-54-2])
piperazinoethyldiethylenetriamines (PEDETA) (CAS No. [31295-49-5]).

Further compounds and isomers may be formed to a minor extent, but alongside the compounds mentioned explicitly above their sum in quantity terms is less than 3% by weight relative to the weight of the technical TETA or TEPA. It is possible to employ a mixture of TETA and TEPA, or TETA alone or TEPA alone.

Both polyamines, TETA and TEPA, have a high N/C ratio and, accordingly, it is possible to incorporate a quantity of amino groups by means of a small molecule and, accordingly, with a small amount.

The Mannich base prepared from resorcinol, formaldehyde and TETA and/or TEPA preferably has an amino number of between 800 and 1100 mg/g KOH, more particularly in the range between 900 and 1000 mg/g KOH, preferably in the range between 950 and 1000 mg/g KOH. It is possible to prepare Mannich bases which no longer contain any measurable amounts of unreacted resorcinol.

A further aspect of the present invention relates to the preparation of the Mannich base described.

For this preparation, resorcinol, triethylenetetramine and/or tetraethylenepentamine are reacted with formaldehyde at a temperature of below 25° C. In particular, formaldehyde is added with stirring and cooling, to a temperature below 25° C., more particularly below 15° C., to a premix of resorcinol and triethylenetetramine and/or tetraethylenepentamine. The addition is made preferably in portions, more particularly in the form of dropwise addition. The resorcinol/[TETA and/or TEPA] premix is preferably heated initially to a temperature of approximately 80° C., in order to dissolve the resorcinol, and is cooled further before the formaldehyde is added. It has emerged as being advantageous if the premix further comprises a solvent, more particularly an alcohol, preferably methanol, in order to dissolve the resorcinol more effectively and in order to lower the viscosity, this solvent being added before the reaction with formaldehyde. With particular advantage the solvent is used right at the start, i.e. during the preparation of the premix. Following the dropwise addition of the formaldehyde, the reaction mixture is preferably increased to a temperature, and more particularly to about 95° C., and at the same time a slight vacuum of typically 0.6 to 0.9 bar is applied. Under these conditions the water introduced by the formaldehyde, possibly, and also the water formed and also any solvent used, are distilled off. The solvent used, accordingly, should advantageously be selected such that it is easy to distill off at this temperature and pressure.

It has been found particularly advantageous that, even without the presence of additional tertiary amines not already present in technical triethylenetetramine and/or technical tetraethylenepentamine, the Mannich bases can be prepared.

The molar ratios of resorcinol to formaldehyde to the sum of TETA and TEPA are in particular 1:1.5-2.5:2.5-3.5. A figure of 1 to about 2 to about 3 for such a ratio has proved particularly suitable.

The Mannich base thus formed is preferably free from unreacted resorcinol, which means that there are no measurable amounts of resorcinol in the Mannich base, and it has in particular an amine number of between 800 and 1100 mg/g KOH, more particularly in the range between 900 and 1000 mg/g KOH, preferably in the range between 950 and 1000 mg/g KOH.

The Mannich base can be used as it is or in a composition.

The Mannich bases are suitable in particular as curing agents for an amine-reactive substance which has at least two amine-reactive functional groups. Particularly suitable amine-reactive functional groups of this kind are glycidyl ether groups and/or isocyanate groups.

In one embodiment the amine-reactive substance which has at least two amine-reactive functional groups is a diglycidyl ether. More particularly it is a diglycidyl ether of bisphenol A, bisphenol F or bisphenol A/F. With particular preference a diglycidyl ether of this kind is what is called a liquid resin, particularly of the kinds available on the market under the trade name Araldite® GY 250, Araldite® PY 304, Araldite® GY 282 (Huntsman) or D.E.R 331 (Dow).

In another embodiment the amine-reactive substance which has at least two amine-reactive functional groups is a polyisocyanate or a prepolymer containing at least two isocyanate groups. Suitable polyisocyanate is more particularly 1,6-hexamethylene diisocyanate (HDI), 2,2,4- and 2,4,4-trimethyl-1,6-hexamethylene diisocyanate (TMDI), 1-isocyanato-3,3,5-trimethyl-5-isocyanatomethylcyclohexane (i.e. isophorone diisocyanate or IPDI), 2,4- and 2,6-tolylene diisocyanate (TDI), and 4,4′-, 2,4′- and 2,2′-diphenylmethane diisocyanate (MDI). Prepolymer containing at least two isocyanate groups comprises, in particular, prepolymers of the kind obtainable from at least one of the aforementioned polyisocyanates and at least one polyol. Suitable polyols include, in particular, polyoxyalkylene polyols or polyester polyols with at least two OH groups, more particularly with 2 or with 3 OH groups.

Mixing of the amine-reactive substance which has at least two amine-reactive functional groups with the Mannich base of the invention produces a reaction of the aminic groups of the Mannich bases with the amine-reactive functional groups of the amine-reactive substance, and curing takes place.

Accordingly the present invention also comprises a two-component composition composed of a first component K1 and a second component K2. The first component K1 comprises at least one amine-reactive compound having at least two functional groups which can react with amines. The second component K2 comprises at least one Mannich base of the kind already described above. The compounds suitable as amine-reactive compounds containing at least two functional groups which can react with amines have already been described above.

The first component, K1, advantageously comprises a plurality of amine-reactive compounds. Thus, in particular, the use of a relatively high-viscosity amine-reactive compound and of a low-viscosity amine-reactive compound is recommended. As the low-viscosity amine-reactive compound particular preference is given to what are known as reactive diluents.

Besides the Mannich base, the second component, K2, can comprise further amines. The amines in question are in particular an aliphatic or cycloaliphatic amine, preferably isophoronediamine (IPDA). Component K2 may further preferably comprise TETA or TEPA. This additional amine can be added as early as at the end of the Mannich base or not until component K2 is being formulated.

Both components, K1 and K2, may if needed comprise further ingredients known to the skilled worker. Further ingredients of this kind are, more particularly, fillers, plasticizers, solvents, catalysts and/or additives.

Preferred fillers include, in particular, carbon blacks, chalks, especially coated chalks, sands, silicates, light-weight fillers, such as ceramic beads or glass beads, more particularly hollow ceramic or glass beads, fumed silicas, and flyash.

Preferred solvents are, in particular, solvents of the kind not classed as VOCs, volatile organic compounds. Relatively high-boiling hydrocarbons are particularly preferred.

Suitable plasticizers are, in particular, phthalates and adipates, more particularly diisodecyl phthalate (DIDP) and dioctyl adipate (DOA).

The uses of two-component compositions of this kind are broad. Particular preference is given to their use as an adhesive or sealant, more particularly as a structural adhesive. It has been found, indeed, that the properties which were achievable by means of the Mannich bases of the invention are particularly desirable in the adhesives segment especially.

In particular it has been found that high cure rates, especially at low temperatures, can be achieved, and that high glass transition temperatures (Tg) can be attained, even when curing takes place cold, i.e., at room temperatures. This is particularly important for epoxy resin compositions, since Mannich base-free amine curing agents used to date to achieve high TGs, examples being isophoronediamine-based curing agents, either have had to be reacted at high temperatures, i.e., above 60° C., or have necessarily involved, after room-temperature curing, a subsequent heating operation, i.e., subsequent heating to temperatures of above 60° C. Moreover, with epoxy resins reacted with Mannich base-free amine curing agents, a problem which often arises is that the curing remains at the so-called beta stage and the ultimate strength is attainable only through subsequent heating. Furthermore, Mannich base-free amine curing agents of this kind have been very difficult, if not impossible, to cure at temperatures below 10° C., more particularly below 5° C. These disadvantages of the prior art can be eliminated by Mannich bases of the invention. In particular, after curing at room temperature, glass transition temperatures of more than 80° C. are achievable without the need for subsequent heating. Moreover, compositions of this kind cure even at low temperatures, in particular below 10° C., preferably between −10° C. and 5° C.

For all applications it is important, not least on ecotoxicological and occupational hygiene grounds, that, with the Mannich bases of the invention, it is possible to provide curing components which are free from phenols, but also free from other phenolic compounds, and preferably are also free—that is, do not any longer contain measurable amounts—of unreacted resorcinol.

After components K1 and K2 of the two-component composition described have been mixed, the adhesive is applied to a substrate surface and joined to a further substrate surface. The cured composition acts as an adhesive layer which is capable of transferring forces between the two substrate surfaces of the composite formed.

On account of its properties, the two-component composition is especially suitable as a structural adhesive in construction, civil engineering, and industry.

By way of example, a two-component composition of this kind, more particularly a two-component epoxy resin composition, i.e., when component K1 comprises a diglycidyl ether, can be used as an adhesive for the bonding of fiber-reinforced composites. An illustrative example of this is the bonding of carbon fiber strips in the reinforcement of constructions, such as bridges.

Furthermore, two-component compositions of the invention, more particularly a two-component epoxy resin composition, can be used as a polymeric matrix for the production of fiber-reinforced composites. Thus, for example, carbon fibers or glass fibers can be embedded into a two-component composition and in the cured state can be employed as a fiber composite, in the form of a lamella, for example.

Likewise, for example, woven or laid fiber fabrics can be applied to a construction by means of a two-component composition, more particularly by means of a two-component epoxy resin composition, and there, together with the construction, form a fiber-reinforced composite.

### EXAMPLES

### Preparation of Mannich Bases

### a) With Solvent Dilution

1 mol of the phenolic compound specified in Table 1 was charged under nitrogen to a reaction vessel, together with 3 mol of the technical-grade polyamine specified in Table 1 and 90 g of methanol, and heating took place if necessary, up to a maximum of 80° C., until the phenol dissolved. Subsequently an ice bath was used for cooling to a temperature between 2 and 13° C. Then, with stirring, 2 mol of formaldehyde (used in the form of 37% formalin solution) was added dropwise with intensive stirring and ice-bath cooling. Following the complete dropwise addition of the formaldehyde, the temperature was slowly raised to 90° C. under a pressure of 400 mbar. Finally, the vacuum was increased to 50 mbar. The quantity of distillate obtained corresponds to the amount of the theoretical amount of methanol and water which was used and has formed.

TABLE 1
Properties of Mannich bases (variation of phenolic compound).

| | M1 | Ref. M1 | Ref. M2 |
|---|---|---|---|
| Phenolic | resorcinol | 3,5-xylenol | m-cresol |
| compound | | | |
| Grade, | distilled flakes, | purum, Fluka | ≧99%, Fluka |
| supplier | Clariant | (Switzerland) | (Switzerland) |
| | (Switzerland) | | |
| Polyamine | TETA | TETA | TETA |
| Grade, | technical, | technical, | technical, |
| supplier | Fluka | Fluka | Fluka |
| | (Switzerland) | (Switzerland) | (Switzerland) |
| Residual | 19.4 ± 1% | 31.3 ± 1.3% | 34.1 ± 0.3% |
| polyamine | | | |
| content* | | | |
| Residual | 0%** | 12.52 ± 1.3% | 15.11 ± 0.05% |
| phenolic | | | |
| compound | | | |
| content* | | | |
| Viscosity | 29 100 | 348 | 206 |
| [mPas] | | | |
| Amine number | 986 | 879 | 921 |
| [mg/g KOH] | | | |

*Average of a threefold determination
**Detection limit below 0.05%

The viscosity was measured by means of a cone/plate Rheomat at 20° C. (40 mm cone, 300 revolutions/s, or 20 mm cone, 50 revolutions/s).

The residual polyamine content was determined by means of GC/FID (Optima-5MS, 60 mg dissolved in 10 ml of ethyl acetate, carrier gas He, external 3-point calibration in the concentration range 2-6 mg/ml) and the residual phenolic compound content was determined by means of HPLC/PDA (Varian, LiChrosphere 100 RP-18, eluent water, acetonitrile, UV 273 nm).

The amine number was determined by titrimetry on a Mettler, Switzerland, Memotitrator DL-55.

b) without Solvent Dilution

1 mol of resorcinol was charged under nitrogen to a reaction vessel together with 1.7 mol of the polyamine specified in Table 2, in technical grade, and this initial charge was heated to 140° C. until solid resorcinol was no longer present. Then, using an ice bath, the charge was cooled to a temperature between 90° C. and 80° C., followed by addition of a further 1.7 mol of the polyamine specified in Table 2, in technical grade, and by cooling to a temperature between 5° C. and 10° C. Subsequently, with stirring, 2 mol of formaldehyde (used in the form of 37% formalin solution) were added dropwise with intensive stirring and ice bath cooling. Following the complete dropwise addition of the formaldehyde, the temperature was raised slowly to 150° C. under a pressure of 400 mbar. Finally the vacuum was increased to 50 mbar. The amount of distillate obtained corresponds to the amount of the theoretical amount of the water which was used and has formed. The result in each of M2 and M3 was a stable Mannich base, whereas the reference examples with IPDA (Ref. M3), diethylenetriamine (DETA) (Ref. M4) as polyamine underwent gelling.

TABLE 2
Mannich bases variation of the polyamine.

| | M2 | M3 | Ref. M3 | Ref. M4 |
|---|---|---|---|---|
| Phenolic | Resorcinol | Resorcinol | Resorcinol | Resorcinol |
| compound | | | | |
| Grade, | distilled | distilled | distilled | distilled |
| supplier | flakes, | flakes, | flakes, | flakes, |
| | Clariant | Clariant | Clariant | Clariant |
| | (Switzerland) | (Switzerland) | (Switzerland) | (Switz- |
| | | | | erland) |
| Polyamine | TETA | TEPA | IPDA | DETA |
| Grade, | technical, | technical, | puriss., | ≧98%, |
| supplier | Fluka | Fluka | Fluka | Fluka |
| | (Switzerland) | (Switzerland) | (Switzerland) | (Switz- |
| | | | | erland) |

### Use as Curing Agents

The Mannich bases were used as curing component K2 for an epoxy resin component K1. The epoxy resin component was composed either of 80% by weight of diglycidyl ether of bisphenol A (Araldite® GY 250, Huntsman) and 20% by weight of hexanediol diglycidyl ether (Araldite® DY-H, Huntsman, epoxy number 6.25-6.65) (K1-1) or of 85% by weight of diglycidyl ether of bisphenol A (Araldite® GY 250, Huntsman) and 15% by weight of trimethylolpropane triglycidyl ether (Araldite® DY-T/CH, Huntsman) (K1-2).

TABLE 3
Properties of cured compositions.

| | Z1 | Ref. Z1 | Ref. Z2 | Z2 |
|---|---|---|---|---|
| K1 | K1-2 | K1-2 | K1-2 | K1-1 |
| K2 | M1 | Ref. 1 | Ref. 2 | M3 |
| K1/K2 [g/g] | 80/20 | 80/20 | 80/20 | 80/20 |
| Potlife | 18 | 22 | 20 | 29 |
| TS (1 d) [MPa] | 43.6 | 47.1 | 51.6 | n.d.^{†} |
| BE (1 d) [%] | 1.4 | 1.6 | 1.8 | n.d.^{†} |
| Tg [° C.] | 96 | 108 | 105 | n.d.^{†} |

n.d.^{†} = not determined.

The potlife was determined by stirring 100 g of the mixed components at room temperature in a beaker, using a spatula. The potlife reported was the time at which the batch underwent gelling.

The tensile strength (TS (1d)) and the breaking extension (BE (1d)) were measured after 1 day of curing at room temperature in accordance with ISO 527, with a measuring speed of 5 mm/min on a Zwick tensile strength apparatus.

The glass transition temperature was measured as the peak maximum by means of DSC (0-250° C., 10°/min).

### Use as Adhesive

The compositions Z1 and Z2 were used to bond aluminum plates and steel plates. The adhesives exhibited effective adhesion and effective bond strengths.

In addition, the Mannich base M1, as curing component K2, was mixed with the A component of Sikadur®-30 (available commercially from Sika Schweiz AG), which is based on bisphenol A diglycidyl ether and on epoxy reactive diluent, as component K1, in a mixing ratio of 1:10, and used to bond a concrete slab to a concrete group. The bond exhibited effective adhesion and an effective bond strength.

1. A Mannich base preparable from resorcinol, formaldehyde, and triethylenetetramine and/or tetraethylenepentamine.

2. The Mannich base of claim 1, wherein the resorcinol:formaldehyde:(triethylenetetramine+tetraethylenepentamine) molar ratios are=1:1.5-2.5:2.5-3.5.

3. The Mannich base of claim 1, wherein the triethylenetetramine and/or tetraethylenepentamine is a technical-grade triethylenetetramine and/or tetraethylenepentamine.

4. The Mannich base of claim 1, wherein the amine number is between 800 and 1100 mg/g KOH₂.

5. The Mannich base of claim 1, wherein the Mannich base contains no measurable amounts of unreacted resorcinol.

6. A process for preparing a Mannich base of claim 1, wherein resorcinol, triethylenetetramine and/or tetraethylenepentamine are reacted with formaldehyde at a temperature of below 25° C.

7. The process of claim 6, wherein the reaction takes place without the presence of additional tertiary amines not already present in technical triethylenetetramine and/or technical tetraethylenepentamine.

8. The process of claim 6, wherein formaldehyde is added with stirring to a premix comprising resorcinol and triethylenetetramine and/or tetraethylenepentamine.

9. The process of claim 6, wherein a solvent, is added before the reaction with formaldehyde.

10. The process of claim 6, wherein, after the reaction with formaldehyde, water and any solvent is distilled off.

11. A two-component composition composed of a first component K1 which comprises at least one amine-reactive compound having at least two functional groups which can react with amines, and a second component K2 which comprises at least one Mannich base of claim 1.

12. The two-component composition of claim 11, wherein the first component K1 comprises, in addition to a Mannich base preparable from resorcinol, formaldehyde, and triethylenetetramine and/or tetrethylenepentamine, a further amine.

13. The two-component composition of claim 11, wherein the amine-reactive compound having at least two functional groups which can react with amines in component K1 is a diglycidyl ether.

14. The two-component composition of claim 11, wherein the amine-reactive compound having at least two functional groups which can react with amines in component K1 is a polyisocyanate or a prepolymer containing at least two isocyanate groups.

15. The two-component composition of claim 11 utilized as an adhesive or sealant.

16. A cured composition wherein it is obtained by mixing and curing the two components K1 and K2 of a two-component composition of claim 11.

17. Composite body which has a hardened composition according to claim 16 as an adhesive layer.

18. The Mannich base of claim 1 utilized as a curing agent for an amine-reactive substance which has at least two amine-reactive functional groups.

19. The Mannich base of claim 18, wherein the amine-reactive substance which has at least two amine-reactive functional groups is a diglycidyl ether.

20. The Mannich base of claim 18, wherein the amine-reactive substance which has at least two amine-reactive functional groups is a polyisocyanate or a prepolymer containing at least two isocyanate groups.
